(19) United States
(12) Patent Application Publication
(10) Pub. No.: US 2014/0277416 A1
(43) Pub. Date: Sep. 18, 2014

(54) Seamless Tubular Extracellular Matrix Prosthetic Valve and Method for Forming Same

(71) Applicant: Robert Matheny, Norcoss, GA (US)

(72) Inventor: Robert Matheny, Norcoss, GA (US)

(21) Appl. No.: 13/804,683

(22) Filed: Mar. 14, 2013

(51) Int. Cl. A61F 2/24

(52) U.S. Cl. CPC A61F 2/2403
USPC 623/2.15

(57) ABSTRACT

A seamless prosthetic valve comprising a continuous tubular member having an outer abluminal surface, a triple walled intermediate portion, and at least a first valve leaflet that is configured to selectively restrict fluid flow through the valve, the triple walled intermediate portion being formed by everting a first end of member over the member, whereby a double walled end is formed, and reverting the first end over the double walled end of the member, the first valve leaflet being formed by suturing the triple walled intermediate portion at a first commissure connection point.

### FIELD OF THE INVENTION

The present invention generally relates to prosthetic valves for replacing defective cardiovascular valves. More particularly, the present invention relates to seamless tubular extracellular matrix (ECM) prosthetic valves for replacing defective aortic, pulmonary, mitral, tricuspid and/or peripheral venous valves, and methods for forming same.

### BACKGROUND OF THE INVENTION

As is well known in the art, the human heart has four valves that control blood flow circulating through the human body. On the left side of the heart is the mitral valve, located between the left atrium and the left ventricle, and the aortic valve, located between the left ventricle and the aorta. Both of these valves direct oxygenated blood from the lungs into the aorta for distribution through the body.

The tricuspid valve, located between the right atrium and the right ventricle, and the pulmonary valve, located between the right ventricle and the pulmonary artery, however, are situated on the right side of the heart and direct deoxygenated blood from the body to the lungs.

The peripheral venous system also includes a number of valves that prevent retrograde blood flow. By preventing retrograde blood flow, the valves found throughout the venous system assist the flow of blood through the veins and returning to the heart.

Normally, the mitral valve has two leaflets and the tricuspid valve has at least two, preferably three leaflets. The aortic and pulmonary valves, however, have normally at least two, preferably three leaflets, also often referred to as “cusps” because of their half-moon like appearance.

Venous valves are usually of the bicuspid type, with each cusp or leaflet forming a reservoir for blood, which, under pressure, forces the free edges of the cusps together to permit mostly antegrade blood flow to the heart. As discussed in detail below, since a majority of venous blood flow is against gravity while a person is standing, incompetent or destroyed venous valves can cause significant medical problems in the legs, ankles, and feet.

Valve diseases are typically classified into two major categories: stenosis and insufficiency. In the case of a stenosis, the native valve does not open properly, whereby insufficiency represents the opposite effect showing, deficient closing properties.

Insufficiency of the inlet (atrioventricular) tricuspid valve to the right ventricle of the heart results in regurgitation of blood back into the right atrium, which, serving to receive blood flow returning in the veins from the entire body, then results in turn in suffusion and swelling (edema) of all the organs, most notably in the abdomen and extremities, insufficient forward conduction of blood flow from the right ventricle into the lungs causing compromise of pulmonary function, and ultimately pump failure of the right heart. Collectively these conditions are termed right heart failure, a condition that leads to incapacity and possibly to death if progressive and uncorrected.

Insufficiency of vein function due to the incompetence or destruction of peripheral venous valves leads to acute then chronic swelling of the veins and their dependent lymphatics and tissues. This condition can affect the deep veins of the body, commonly the lower extremities or pelvis, or the superficial veins of the lower extremities in particular, leading to progressive expansion of the veins and further valvular incompetence, a condition known as varicose veins.

Medical conditions like high blood pressure, inflammatory and infectious processes often lead to stenosis and insufficiency. Treatment of heart valve dysfunctions typically include reparation of the diseased heart valve with preservation of the patient's own valve or replacement of the valve with a mechanical or bioprosthetic valve (i.e. “tissue” valve), i.e. a prosthetic valve. Particularly for aortic heart valves, however, it is frequently necessary to introduce a heart valve replacement.

Various prosthetic heart valves have thus been developed for replacement of natural diseased or defective valves. Illustrative are the tubular prosthetic tissue valves disclosed in Applicant's Co-Pending U.S. application Ser. Nos. 13/560,573, 13/782,024 and 13/782,289. A further tubular prosthetic valve is disclosed in U.S. Pat. No. 6,126,686.

A major drawback associated with most tubular prosthetic valves, such as the valves disclosed in U.S. Pat. No. 6,126,686, is that the valves are typically formed from one or more sheets of tissue material, e.g., submucosal tissue, which is initially wrapped around a mandrel to form a tubular structure. The resulting tubular construct thus includes a seam extending the length of the construct, which can, and in many instances will, cause perivalvular leakage.

Various conventional sealing techniques have thus been employed to prevent perivalvular leakage from tubular valve constructs, including suturing, crosslinking, binding with adhesives, etc. Although the noted sealing techniques can be, and most times are, highly effective to seal tubular valve constructs, success of the techniques is highly dependent on the processing techniques and/or processing technician, and/or the skill of the surgeon.

Implantation of a prosthetic valve, including mechanical valves and bioprosthetic valves, also requires a great deal of skill and concentration given the delicate nature of the native cardiovascular tissue and the spatial constraints of the surgical field. It is also critical to achieve a secure and reliable attachment of the valve to host cardiovascular tissue.

Various structures and means have thus also been developed to provide a secure and reliable attachment of a prosthetic valve to host cardiovascular tissue. Most surgical techniques comprise suturing the ends of the valve to the annulus of the cardiovascular vessel.

There are numerous drawbacks and disadvantages associated with suturing a valve to host tissue. A major disadvantage is similarly the high risk of perivalvular leakage.

In application Ser. No. 13/560,573 the tissue valve includes a sewing ring that can be employed to suture the ends of the valve to the annulus of the cardiovascular vessel. Although the use of a sewing ring to secure the valve to a cardiovascular vessel can be, and most times is, highly effective, success of the technique is again still highly dependent on the skill of the surgeon.

There is thus a need to provide “seamless” prosthetic valves that can be readily employed to selectively replace diseased or defective aortic, pulmonary, mitral, tricuspid and peripheral venous valves.

There is also a need to provide prosthetic valves having means for secure, reliable and consistent attachment to cardiovascular vessels.

It is therefore an object of the present invention to provide seamless prosthetic tissue valves that can be readily employed to selectively replace diseased or defective aortic, pulmonary, mitral, tricuspid and peripheral venous valves.

It is another object of the present invention to provide a method for forming seamless prosthetic tissue valves that can be readily employed to selectively replace diseased or defective aortic, pulmonary, mitral, tricuspid and peripheral venous valves

It is another object of the present invention to provide seamless prosthetic tissue valves having means for secure, reliable, and consistently highly effective attachment to cardiovascular vessels.

It is another object of the present invention to provide seamless prosthetic tissue valves that substantially reduce or eliminate intimal hyperplasia after intervention in a vessel and the harsh biological responses associated with conventional polymeric and metal valves.

It is another object of the present invention to provide seamless extracellular matrix (ECM) prosthetic tissue valves that induce host tissue proliferation, bioremodeling and regeneration of new tissue and tissue structures with site-specific structural and functional properties.

It is another object of the present invention to provide seamless extracellular matrix (ECM) prosthetic tissue valves that are capable of administering a pharmacological agent to host tissue and, thereby produce a desired biological and/or therapeutic effect.

### SUMMARY OF THE INVENTION

The present invention is directed to seamless prosthetic tissue valves that can be readily employed to selectively replace diseased or defective aortic, pulmonary, mitral, tricuspid and peripheral venous valves, and methods for forming same.

In a preferred embodiment of the invention, the seamless prosthetic valves comprise continuous tubular members having first and second ends, a triple walled intermediate portion, and at least one internal valve leaflet, the triple walled intermediate portion being formed by everting the first end of the tubular member over the tubular member to form a double walled first end and a doubled wall portion proximal to and extending from said double walled end, and reverting the first end of the tubular member over the double walled end of the tubular member, the internal valve leaflet being formed by suturing the three walls of the triple walled intermediate portion at a first commissure connection point.

In some embodiments, the three walls of the triple walled intermediate portion are sutured at two commissure connection points to form two valve leaflets therein.

In some embodiments, the three walls of the triple walled intermediate portion are sutured at three commissure connection points to form three valve leaflets therein.

In a preferred embodiment of the invention, the tubular member comprises mammalian small intestine submucosa.

In some embodiments, the small intestine submucosa comprises porcine small intestine submucosa.

In some embodiments of the invention, the tubular member (or material thereof) includes at least one additional biologically active agent or composition, i.e. an agent that induces or modulates a physiological or biological process, or cellular activity, e.g., induces proliferation, and/or growth and/or regeneration of tissue.

In some embodiments, the biologically active agent comprises a protein.

In some embodiments, the biologically active agent comprises a cell.

In some embodiments, the tubular member (or material thereof) includes at least one pharmacological agent or composition (or drug), i.e. an agent or composition that is capable of producing a desired biological effect in vivo, e.g., stimulation or suppression of apoptosis, stimulation or suppression of an immune response, etc.

In some embodiments of the invention, the pharmacological agent comprises an anti-inflammatory agent.

In some embodiments of the invention, the pharmacological agent comprises a statin, i.e. a HMG-CoA reductase inhibitor.

In some embodiments of the invention, the seamless prosthetic valves include at least one anchoring mechanism.

In some embodiments of the invention, the anchoring mechanism comprises at least one reinforcing ring or band that is positioned and secured at a desired position on or in the valve.

In some embodiments of the invention, the anchoring mechanism comprises at least two reinforcing rings that are positioned and secured at desired positions, e.g. proximal and distal ends, on or in the valve.

In a preferred embodiment of the invention, the anchoring mechanisms are designed and configured to position the seamless prosthetic valves proximate the wall of a vessel (i.e. host tissue thereof), and maintain contact therewith, for a predetermined temporary support time period.

In some embodiments of the invention, the support time period is within the process of tissue regeneration.

The seamless prosthetic valves of the invention provide numerous advantages compared to prior art prosthetic valves. Among the advantages are the following:
The provision of seamless prosthetic tissue valves that can be readily employed to selectively replace diseased or defective aortic, pulmonary, mitral, tricuspid and peripheral venous valves
The provision of seamless prosthetic tissue valves that substantially reduce or eliminate intimal hyperplasia after intervention in a vessel and the harsh biological responses associated with conventional polymeric and metal valves.
The provision of seamless prosthetic tissue valves that induce host tissue proliferation, bioremodeling and regeneration of new tissue and tissue structures with site-specific structural and functional properties.
The provision of seamless prosthetic tissue valves that are capable of administering a pharmacological agent to host tissue and, thereby produce a desired biological and/or therapeutic effect.
The provision of seamless prosthetic tissue valves that include anchoring mechanisms that temporarily position the valves proximate cardiovascular tissue for a pre-determined period of time.
The provision of seamless prosthetic tissue valves that exhibit optimum mechanical compatibility with vascular structures.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of one embodiment of a tube of submucosal tissue that can be employed to form a seamless prosthetic valve, in accordance with the invention;

FIGS. 2A-2C are perspective sectional views of one embodiment of a seamless prosthetic valve formed from the tube of submucosal tissue shown in FIG. 1, in accordance with the invention;

FIGS. 3A-3B are schematic illustrations showing various valve commissure connection points, in accordance with the invention;

FIG. 4 is a front (or end) plan view of a proximal end of one embodiment of a seamless prosthetic valve, showing the leaflets formed by blood flow (i.e. regurgitating blood) therethrough, in accordance with the invention; and

FIG. 5 is a side plan, partial sectional view of an anchored seamless prosthetic valve, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### DEFINITIONS

The terms “anchoring mechanism” and “anchor”, as used herein in connection with some embodiments of the two-piece anchored valves, mean a temporary structure that is configured and employed to “temporarily” position the valve proximate vessel tissue. As discussed in detail herein, in some embodiments of the invention, the anchoring mechanisms are designed and configured to temporarily position tissue valves proximate a recipient's cardiovascular tissue for a predetermined period of time, which, in some embodiments, is preferably within the process of new tissue regeneration.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “biologically active agent” and “biologically active composition” are used interchangeably herein, and mean and include agent that induces or modulates a physiological or biological process, or cellular activity, e.g., induces proliferation, and/or growth and/or regeneration of tissue.

The terms “biologically active agent” and “biologically active composition” thus mean and include, without limitation, the following growth factors: platelet derived growth factor (PDGF), epidermal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNA-alpha), and placental growth factor (PLGF).

The terms “biologically active agent” and “biologically active composition” also mean and include, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrillar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oxytetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrine, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V antiarrhythmic agents: (Class Ia) quinidine, procainamide and disopyramide; (Class Ib) lidocaine, phenyloin and mexiletine; (Class Ic) flecamide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “pharmacological agent”, “active agent”, “drug” and “active agent formulation” can further include one or more classes of cytotoxic anti-neoplastic agents or chemotherapy agents, including, without limitation, alkylating agents, cisplatin, carboplatin, oxaliplatin, mechlorethamine, cyclophosphamide, chlorambucil, and ifosfamide. Chemotherapy agents can also include, without limitation, antimetabolites, such as purine analogues, pyrimidine analogues and antifolates, plant alkaloids, such as vincristine, vinblastine, vinorelbine, vindesine, podophyllotoxin, etoposide and teniposide, taxanes, such as paclitaxel and docetaxel, topoisomerase inhibitors, such as irinotecan, topotecan, amsacrine, etoposide, etoposide phosphate and teniposide, cytotoxic antibiotics, such as actinomycin, bleomycin, plicamycin, mytomycin and anthracyclines, such as doxorubicin, daunorubicin, valrubicin, idarubicin, epirubicin, and antibody treatments, such as abciximab, adalimumab, alamtuzumab, basiliximab, belimumab, bevacizumab, brentuximab vedotin, canakinumab, cetuximab, certolizumab pego, daclizumab, denosumab, eculizumab, efalizumab, gemtuzamab, golimumab, ibritumomab tiuxetan, infliximab, ipilimumab, muromonab-CD3, natalizumab, ofatumumab, omalizumab, palivizumab, panitumumab, ranibizumab, rituximab, tocilizumab (atlizumab), tositumomab and trastuzumab.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, morniflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sennetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, trifiumidate, zidometacin, and zomepirac sodium.

The term “pharmacological composition”, as used herein, means and includes a composition comprising a “pharmacological agent” and/or a “biologically active agent” and/or any additional agent or component identified herein.

The term “therapeutically effective”, as used herein, means that the amount of the “pharmacological agent” and/or “biologically active agent” and/or “pharmacological composition” administered is of sufficient quantity to ameliorate one or more causes, symptoms, or sequalae of a disease or disorder. Such amelioration only requires a reduction or alteration, not necessarily elimination, of the cause, symptom, or sequalae of a disease or disorder.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As stated above, the present invention is directed to one-piece, seamless prosthetic valves which, in a preferred embodiment, are formed from an extracellular matrix material. According to the invention, the seamless prosthetic valves of the invention can be readily designed and configured and, hence, employed to replace native valves in the body including, without limitation, diseased or defective aortic, pulmonary, mitral, tricuspid and/or peripheral venous valves.

The seamless prosthetic valves of the invention can also be deployed in various cardiovascular vessels by traditional or minimally invasive means.

As discussed in detail herein, in a preferred embodiment, the seamless prosthetic valves comprise continuous tubular members having first and second ends, a triple walled intermediate portion, and at least one internal valve leaflet, the triple walled intermediate portion being formed by everting the first end of the tubular member over the tubular structure to form a double walled first end and a doubled wall portion proximal to and extending from said double walled end, and reverting the first end of the tubular member over the double walled end of the tubular construct, the internal valve leaflet being formed by suturing the three walls of the triple walled intermediate portion at a first commissure connection point.

In some embodiments, the three walls of the triple walled intermediate portion are sutured at two commissure connection points to form two valve leaflets therein.

In some embodiments, the three walls of the triple walled intermediate portion are sutured at three commissure connection points to form three valve leaflets therein.

According to the invention, the tubular member and, hence, seamless prosthetic valves formed therefrom, can comprise various biocompatible materials, including, without limitation, mammalian tissue, e.g., bovine tissue.

In a preferred embodiment of the invention, the tubular member comprises an extracellular matrix (ECM) material.

According to the invention, the ECM material can be derived from various mammalian tissue sources and methods for preparing same, such as disclosed in U.S. Pat. Nos. 7,550,004, 7,244,444, 6,379,710, 6,358,284, 6,206,931, 5,733,337 and 4,902,508 and U.S. application Ser. No. 12/707,427; which are incorporated by reference herein in their entirety. The mammalian tissue sources include, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

In a preferred embodiment of the invention, the tubular member comprises porcine small intestine submucosal tissue.

As stated above, in some embodiments of the invention, the tubular member (or material thereof) includes at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells and proteins.

In some embodiments, the tubular member (or material thereof) includes at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

In some embodiments of the invention, the pharmacological agent comprises an anti-inflammatory agent.

In some embodiments of the invention, the pharmacological agent comprises a Stalin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor®, Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. The properties and beneficial actions are set forth in Applicant's Co-Pending application Ser. No. 13/373,569, filed on Sep. 24, 2012 and Ser. No. 13/782,024, filed on Mar. 1, 2013; which are incorporated by reference herein in their entirety.

In some embodiments of the invention, the pharmacological agent comprises chitosan. As also set forth in detail in Co-Pending application Ser. No. 13/573,569, chitosan also exhibits numerous beneficial properties that provide several beneficial biochemical actions or activities.

As also indicated above, in some embodiments of the invention, the seamless prosthetic valves of the invention further include at least one anchoring mechanism that is configured to position the valves proximate cardiovascular tissue, and maintain contact therewith for a pre-determined anchor support time period. According to the invention, the anchoring mechanisms can comprise various forms and materials.

In some embodiments of the invention, the anchoring mechanisms comprise reinforcing rings or bands that are positioned and secured at desired positions, e.g. proximal and distal ends, on or in a seamless prosthetic valve. According to the invention, the reinforcing rings and bands preferably comprise a biocompatible material, such as a biocompatible metal, e.g., Nitinol® and stainless steel, and various polymeric materials. The reinforcing rings and bands can also comprise various biodegradable materials, such as magnesium and ECM material.

As defined above and discussed in detail in Co-pending application Ser. No. 13/782,024, the terms “anchoring mechanism” and “anchor”, as used in connection with some embodiments of anchored seamless prosthetic valves of the invention mean a structure that is configured and employed to temporarily position and support a seamless prosthetic valve of the invention proximate host tissue of a vessel.

In some embodiments, the anchoring mechanisms position the anchored seamless valves proximate host tissue of a vessel, and maintain contact therewith for a predetermined temporary anchor support period of time within the process of tissue regeneration.

Referring now to FIGS. 1 and 2A-2C, one embodiment of a seamless prosthetic tissue valve and method for forming same will be described in detail.

As illustrated in FIGS. 1, 2A and 2C, the seamless prosthetic valve 10 comprises a continuous tubular member 11 having an outer surface 13, an inner surface 15, and first and second ends 12, 14, a triple walled intermediate portion 20, and at least one internal valve leaflet that is configured to selectively prevent undesired regurgitation of blood through the valve structure.

In a preferred embodiment, the tubular member 11 is processed as follows: all cellular remnants, e.g., serosa, subserosa, thick muscle layers, etc., are removed from the tubular member 11, which results in a rougher outer surface 13, i.e. abluminal surface, and a smoother inner surface 15; the smoother inner surface 15 resulting from the removal of the tunica mucosa.

Applicant has found that the rough abluminal surface 13 of the tubular member 11 readily attaches to itself and, hence, facilitates effective formation of the two walled end 16 and three walled intermediate portion 20 of the formed valve structure, which is discussed below.

The smooth inner surface 15 of the tubular member 11 will also be less thrombotic and exhibit enhanced endothelialization.

According to the invention, the triple walled intermediate portion 20 is formed by everting the first end of the tubular member 14 over the tubular structure 11, whereby the abluminal surface 13 is in contact with itself and a double walled first end 16 and a doubled wall portion 18 proximal to and extending from said double walled end 16 is formed, and reverting the first end of the tubular member 14 over the double walled end 16 of the tubular member 14.

As indicated above, the seamless prosthetic valve 10 further includes at least one internal valve leaflet. According to the invention, the valve leaflet is formed by suturing the three walls of the triple walled intermediate portion 20 at a first commissure connection point 22a.

In some embodiments, the three walls of the triple walled intermediate portion are sutured at two commissure connection points (denoted “22a” and “22b” in FIG. 3A) to form two valve leaflets therein.

In some embodiments, the three walls of the triple walled intermediate portion 20 are sutured at three, preferably, equally spaced commissure connection points (denoted “22a”, “22b” and “22c” in FIG. 3B) to form three valve leaflets therein (denoted “30”, “32” and “34” in FIG. 4).

According to the invention, the leaflets 30, 32, 34 can have various shapes and sizes, such as shown in U.S. Pat. No. 8,257,434 and Co-pending application Ser. No. 13/560,573, which are incorporated by reference herein.

The size and shape each leaflet 30, 32, 34, i.e. valve structure, is, of course, dependent upon the commissure connection points, i.e. length of double walled end 16 to the commissure connection point(s) (denoted “L” in FIG. 2C) and the size, i.e. operative diameter (denoted “D” in FIG. 2A), of the first member 12 member and, hence, valve structure formed therefrom.

According to the invention, the size or operative diameter “D” and length of the prosthetic valves of the invention can vary to accommodate placement in various adult and pediatric cardiovascular vessels

In some embodiments, the edge length of each leaflet 30, 32, 34 ranges from approximately 10 mm to approximately 70 mm, more preferably from approximately 15 mm to approximately 60 mm, and most preferably from approximately 25 mm to approximately 45 mm. In this aspect, it is contemplated that the ratio between the edge length of each leaflet to the diameter of a target annulus can range from approximately 0.5:1 to approximately 3:1, and more preferably from approximately 1:1 to approximately 2:1. In addition to the noted ratios serving as the endpoints of the ranges set forth above, the disclosed ranges also include all ratios falling between the endpoint ratios.

Referring now to FIG. 5, in some embodiments of the invention, the seamless prosthetic valve 40 further includes at least one anchoring mechanism, more preferably, two anchoring mechanisms 42a, 42b.

As illustrated in FIG. 5, in some embodiments, the anchoring mechanisms 42a, 42b comprise reinforcing rings or bands, which, in the illustrated embodiment, are positioned and secured at proximal 44a and distal 44b ends on the seamless prosthetic valve 40.

According to the invention, the anchoring mechanism 42a, 42b can be disposed at other positions in or on the prosthetic valve 40.

As set forth in detail in Co-pending application Ser. No. 13/782,024, the anchoring mechanisms 42a, 42b are designed and configured to position the seamless prosthetic valve 40 proximate host tissue of a vessel, and maintain contact therewith for a predetermined anchor support period of time.

As will readily be appreciated by one having ordinary skill in the art, the present invention provides numerous advantages compared to prior art prosthetic valves. Among the advantages are the following:
The provision of seamless prosthetic tissue valves that can be readily employed to selectively replace diseased or defective aortic, pulmonary, mitral, tricuspid and peripheral venous valves
The provision of seamless prosthetic tissue valves that substantially reduce or eliminate intimal hyperplasia after intervention in a vessel and the harsh biological responses associated with conventional polymeric and metal valves.
The provision of seamless prosthetic tissue valves that induce host tissue proliferation, bioremodeling and regeneration of new tissue and tissue structures with site-specific structural and functional properties.
The provision of seamless prosthetic tissue valves that are capable of administering a pharmacological agent to host tissue and, thereby produce a desired biological and/or therapeutic effect.
The provision of seamless prosthetic tissue valves that include anchoring mechanisms, which temporarily position the valves proximate cardiovascular tissue for a pre-determined period of time.
The provision of seamless prosthetic tissue valves that exhibit optimum mechanical compatibility with vascular structures.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A seamless prosthetic valve, comprising:
a continuous tubular member having an outer abluminal surface, first and second ends, a triple walled intermediate portion, and at least a first valve leaflet, said first valve leaflet being configured to selectively restrict fluid flow through said seamless prosthetic valve,
said triple walled intermediate portion being formed by everting said first end of said tubular member over said tubular member to form a double walled first end and a doubled wall portion proximal to and extending from said double walled end, and reverting said first end of said tubular member over said double walled end of said tubular member,
said first valve leaflet being formed by suturing said triple walled intermediate portion at a first commissure connection point.

2. The prosthetic valve of claim 1, wherein said triple walled intermediate portion is sutured at two commissure connection points, and wherein said first valve leaflet and a second valve leaflet are formed.

3. The prosthetic valve of claim 1, wherein said triple walled intermediate portion is sutured at three commissure connection points, and wherein said first valve leaflet, and second and third valve leaflets are formed.

4. The prosthetic valve of claim 1, wherein said tubular member comprises an extracellular matrix (ECM) material derived from a mammalian tissue source.

5. The prosthetic valve of claim 4, wherein said tissue source comprises small intestine submucosa.

6. The prosthetic valve of claim 5, wherein said small intestine submucosa comprises porcine small intestine submucosa.

7. The prosthetic valve of claim 4, wherein said ECM material includes an additional biologically active agent.

8. The prosthetic valve of claim 7, wherein said biologically active agent is selected from the group consisting of human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogenic cells, and post-natal stem cells

9. The prosthetic valve of claim 7, wherein said biologically active agent comprises a growth factor selected from the group consisting of a platelet derived growth factor (PDGF), epidennal growth factor (EGF), transforming growth factor alpha (TGF-alpha), transforming growth factor beta (TGF-beta), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNA-alpha), and placental growth factor (PLGF).

10. The prosthetic valve of claim 4, wherein said ECM material includes an additional pharmacological agent.

11. The prosthetic valve of claim 10, wherein said pharmacological agent comprises an anti-inflammatory.

12. The prosthetic valve of claim 10, wherein said pharmacological agent comprises a statin.

13. The prosthetic valve of claim 12, wherein said statin is selected from the group consisting of atorvastatin, cerivastatin, fluvastatin, lovastatin, mevastatin, pitavastatin, pravastatin, rosuvastatin and simvastatin.

14. The prosthetic valve of claim 10, wherein said pharmacological agent comprises an anti-arrhythmic agent.

15. The two-piece valve of claim 14, wherein said anti-arrhythmic agent is selected from the group comprising quinidine, procainamide, disopyramide, lidocaine, phenyloin, mexiletine, flecaimide, propafenone, moricizine, propranolol, esmolol, timolol, metoprolol, atenolol, amiodarone, sotalol, ibutilide, dofetilide, verapamil, diltiazem, adenosine and digoxin.
